(19) United States
(12) Patent Application Publication
(10) Pub. No.: US 2002/0062490 A1
(43) Pub. Date: May 23, 2002

(54) Hypertension

(76) Inventors: Walter J. Koch, Durham, NC (US); Andrea D. Eckhart, Durham, NC (US)

Correspondence Address: NIXON & VANDERHYE P.C., 8th Floor, 1100 North Glebe Road, Arlington, VA, 22201, US

(21) Appl. No.: 09/984,869

(22) Filed: Oct. 31, 2001

Related U.S. Application Data
(60) Provisional application No. 60/244,212, filed on Oct. 31, 2000.

(51) Int. Cl. A01K 67/027

(52) U.S. Cl. 800/9; 800/18

(57) ABSTRACT

The present invention relates, in general, to hypertension and, in particular, to an animal model of hypertension that elucidates a novel therapeutic target for lowering blood pressure.

This application claims priority from Provisional Application No. 60/244,212, filed Oct. 31, 2000, the entire content of which is incorporated herein by reference.

### TECHNICAL FIELD

The present invention relates, in general, to hypertension and, in particular, to an animal model of hypertension that elucidates a novel therapeutic target for lowering blood pressure.

### BACKGROUND

High blood pressure or hypertension is a prevalent disease in the United States and is a leading cause of morbidity and mortality. Understanding the mechanisms that lead to hypertension is critical to improve existing health care for this disorder.

A functional abnormality leading to hypertension can reflect an imbalance between G protein-coupled receptor (GPCR) mediated vasoconstriction and vasodilation. GPCR signaling is mediated via heterotrimeric G proteins which, following receptor activation, dissociate and release activated Gα and Gβγ subunits, both of which are capable of molecular signaling (Post et al, FASEB J. 10:741-749 (1996)). One G protein family member, Gq, is activated by several GPCRs mediating vasoconstriction and its activation can dramatically increase systemic vascular resistance (SVR), mean arterial pressure (MAP) and myocardial and vascular hypertrophy. Activation of another member of the G protein family, Gs, by GPCRs including β-adrenergic receptors (ARs), can mediate vasodilation and a decrease in SVR and MAP. Impairment in βAR-mediated vasodilation due to an alteration in receptor/G-protein coupling increases SVR and has been described to occur in both human and animal models of hypertension (Feldman, J. Clin. Invest. 85:647-652 (1990); Feldman et al, Hypertension 26:725-732 (1995)). This defective βAR coupling is accompanied by selective increases in expression and activity of the G protein-coupled receptor kinase 2 (GRK2 or βARK1) which has been found in the lymphocytes of hypertensive patients (Gros et al, J. Clin. Invest. 99:2087-2093 (1997)). GRK2 phosphorylates and desensitizes agonist-occupied GPCRs, including BARs, and therefore prevents activation of G proteins. Of the 6 members of the GRK family, GRK2, GRK3, GRK5 and GRK6 have been found in VSM (Gros et al, J. Clin. Invest. 99:2087-2093 (1997); Ishizaka et al, J. Biol. Chem. 272:32482-32488 (1997)). Although none of these kinases show substrate selectivity in vitro, recent evidence suggests that in vivo in the heart they show specific actions (Eckhart et al, Circ. Res. 86:43-50 (2000); Koch et al, Ann. Rev. Physiol. 62:237-260)). With respect to important vascular GPCRs, GRK2 desensitizes the β₂AR and angiotensin II receptor in vivo, whereas it is ineffective on the α_{1B}AR (Eckhart et al, Circ. Res. 86:43-50 (2000); Koch et al, Ann. Rev. Physiol. 62:237-260)).

### SUMMARY OF THE INVENTION

The present invention relates to an animal model of hypertension that elucidates a novel therapeutic target for lowering blood pressure.

Objects and advantages of the present invention will be clear from the description that follows.

### BRIEF DESCRIPTION OF THE DRAWINGS

FIGS. 1A and 1B: mRNA expression in two independent lines of transgenic mice overexpressing GRK2 directed by the vascular smooth muscle specific SM22α promoter. FIG. 1A, Schematic of the transgene used to generate GRK2 overexpressing transgenic mice. Arrows indicate location of annealing for primers used for PCR. FIG. 1B, RT-PCR results illustrating expression of transgene in various tissues in BK10 and BK25 mice whereas there is no product present in the NLC mice.

FIGS. 2A-2C: GRK2 expression and activity in transgenic mice. FIG. 2A, Immunoprecipitation and subsequent immunoblot of GRK2 from 1500 μg of cytosolic protein from brain, liver and kidney of BK10 and NLC mice. FIG. 2B, Immunoprecipitation and immunoblot of GRK2 from 250 μg of cytosolic protein isolated from primary cultured aorta and vena cava cells isolated from BK25 mice. FIG. 2C, GRK2 activity in 200 μg of cytosolic protein isolated from primary cultured aorta and vena cava cells.

FIG. 3: Conscious Mean Arterial Pressure (MAP) in NLC versus GRK2 overexpressing (SM22BK10) and GqI expressing (SM22GqI) mice. Blood pressure measurements were made using a left carotid catheter that was tunneled subcutaneously through to the back. At the time of blood pressure measurement, the mice were conscious and moving freely in their cages. The investigator was blinded to the genotype of the animals. n-size is as noted. *p<0.05 versus NLC, unpaired, two-tailed Student's t-test.

FIG. 4: Aorta wall thickness in NLC mice versus mice overexpressing GRK2. Wall thickness was determined at 11 different points along the aorta width and measured in μm. The measurements were then averaged. The investigator was blinded to the genotype of the animals. *p<0.05 versus NLC, unpaired, two-tailed Student's t-test.

FIGS. 5A-5D: Anesthetized blood pressure measurements in response to various GPCR agonists. FIG. 5A, The % increase in MAP as compared to resting MAP with cumulative increasing doses of angiotensin II (Ang II) in NLC and two independent lines of GRK2 overexpressing mice, BK10 and BK25. FIG. 5B, The % increase in MAP as compared to resting MAP with cumulative increasing doses of phenylephrine (PE) in NLC versus BK10 and BK25 mice. FIG. 5C, % decrease in MAP as compared to resting MAP with cumulative increasing doses of isoproterenol (ISO). FIG. 5D, % change from resting diastorlic pressure with 0.625 mg/kg ISO in NLC and BK10 mice, n=9, 4,4 for NLC, BK10 and BK25 respectively, *p<0.05 versus NLC.

FIGS. 6A-6C: Expression of the GRK2 transgene. (FIG. 6A) Expression of the GRK2 transgene was determined by RT-PCR for the SV40 portion of the construct from various tissues isolated from NLC and 2 different lines of SM22□ transgenic mice. (FIG. 6B) In vivo protein levels of GRK2 were determined using immunoblotting techniques. The medial smooth muscle layer from 10 aortas were pooled, protein was extracted and resolved on a 12% SDS-PAGE gel. The (+) lane is protein isolated from the heart of transgenic mice with cardiac overexpression of GRK2. (FIG. 6C) Histogram of arbitrary densitometry units for the immunoblot shown in (FIG. 6B).

FIGS. 7A-7D: In vitro expression and activity of vascular GRK2 overexpression in transgenic mice. (FIG. 7A) Primary cell cultures were established from thoracic aorta and vena cava (VC) of NLC and the two lines of GRK2 overexpressing mice and GRK2 protein levels were assessed by protein immunoblot analysis. (FIG. 7B) Functional expression of the transgene was assessed by determining adenylyl cyclase activity in response to increasing doses of isoproterenol in aorta VSM cells isolated from NLC (◯, n=4) and SM22α-GRK2-25 (●, n=7) mice. Conversion of [³H]adenine to [³H]cAMP was determined. The entire isoproterenol dose-response curve was statistically different in transgenic cells compared with NLC cells. Data are shown as mean±SEM. *P<0.05, two-way ANOVA. (FIG. 7C) ERK1/2 and JNK1/3 activity in response to βAR stimulation in NLC and GRK2-25 aorta smooth muscle. Autoradiogram of an experiment in which γ³²P-ATP-mediated phosophorylation of myeline basic protein (MBP) due to ERK1/2 activity or GST-c-jun due to JNK1/3 activity was assessed in 1 mg of cytosolic fraction isolated from cultured NLC and GRK2-25 aorta cells following stimulation for 5 minutes with increasing doses of isoproterenol (ISO). (FIG. 7D) Histogram of averaged data for ERK1/2 activity in NLC (n=4) and GRK2-25 cells (n=4) in response to stimulation with 10-5M ISO for 5 minutes. Data indicate that there is a functional overexpression of GRK2 in vascular smooth muscle cells isolated from aorta of transgenic mice since the adenylyl cyclase and MAPK activation is attenuated in SM22α-GRK2 cells as compared to NLC cells. Data shown as mean±SEM, *P<0.05 unpaired Student's t-test.

FIGS. 8A-8C: Response of thoracic aorta rings to isoproterenol and phenylephrine. (FIG. 8A) 25 mm segments of thoracic aorta were isolated from NLC (◯, n=3) and SM22-GRK2-25 (●, n=4) and hung on isolated ring baths. Cumulative dose response to isoproterenol was determined in the presence of endothelial cells. *P<0.05 vs NLC, two-way ANOVA. (FIG. 8B) Response of aorta rings to isoproterenol following pretreatment with L-NAME to prevent nitric oxide release from endothelial cells. Response of rings to isoproterenol following mechanical scraping of endothelial cells using a fine wire was also done and the results were similar to L-NAME pretreatment. *P<0.05 vs NLC, two-way ANOVA. (FIG. 8C) The vasoconstrictor phenylephrine increased tension in aorta rings in a similar dose-dependent manner for both NLC (◯, n=3) and SM22α-GRK2 mice (●, n=4).

FIGS. 9A-9D: Conscious blood pressure in NLC and SM22α-GRK2 mice. A fluid-filled indwelling catheter was inserted into the left common carotid artery to determine blood pressure on conscious unrestrained mice 24 hours following catheter insertion. (FIG. 9A) Systolic and (FIG. 9B) diastolic pressures were measured and (FIG. 9C) MAP was calculated from n=9 mice for NLC, SM22α-GRK2-10 (n=7) and SM22α-GRK2-25 mice (n=5). *P<0.05 vs NLC, unpaired t-test. (FIG. 9D) At doses of ISO low enough not to effect heart rate, there was a significant attenuation in the increase in diastolic pressure as compared to resting diastolic pressure in SM22α-GRK2 mice (▪) (n- 3-5) versus NLC mice (□) (n=3-5). There was no difference in resting heart rate between NLC mice (192±28 bpm) and SM22α-GRK2 mice (239±17 bpm). *P<0.05 for transgene effect in two-way ANOVA comparison.

FIGS. 10A-10C: Aorta wall thickness in GRK2 overexpressing mice. Mice were perfusion fixed at 100 mmHg with 10% neutral-buffered formalin. Thoracic aorta were then isolated from (FIG. 10A) NLC and (FIG. 10B) SM22α-GRK2-10 mice, fixed, paraffin embedded and sliced. The slides were then stained using a Verhoeff Van Gieson/Masson's protocol. A 20 μm length is represented by the bar in the NLC panel and the same scale was used for each aorta. (FIG. 10c) Histogram of the aorta wall thickness for SM22α-GRK2 mice (▪) (n=6) compared with NLC mice (□) (n=7). *P<0.05 vs NLC, unpaired t-test.

FIG. 11: Myocardial hypertrophy in GRK2 overexpressing mice. Heart weight-to-body weight ratio (mg/g) in NLC (n=7) and both lines of GRK2 overexpressing mice combined (n=10). *P<0.05 vs NLC, unpaired t-test.

### DETAILED DESCRIPTION OF THE INVENTION

Since GRK2 or βARK1 protein levels and activity are increased in hypertensive patients and whereas no changes in GRK5/6 were described (Gros et al, J. Clin. Invest. 99:2087-2093 (1997)), it was hypothesized that this could be a primary defect responsible for the pathogenesis of high blood pressure. The increase GRK activity as seen in human hypertensives could potentially upset the fine balance establishing blood pressure and SVR. Therefore a transgenic mice overexpressing GRK2 (βARK1) in a vascular smooth muscle (VSM) specific manner was created. This was done by using a segment of the SM22α promoter which had been previously shown to be an arterial SMC marker, however, besides marker transgenes no one had previously developed transgenic mice expressing a functional transgene.

The entire coding region for bovine GRK2 was linked to this promoter and several lines of mice were generated. Two of these lines have been characterized to date and it has been found first that these mice overexpress GRK2 in both their arterial and venous VSM and that the amount of GRK2 overexpression (3-5 fold) is similar to the overexpression levels found in human hypertensive samples. Upon testing, it was found that by only manipulating GRK2 levels and GRK activity in the VSM of these mice, this led to significant in vivo resting high blood pressure. These mice appear to have a complete hypertension syndrome as the arteries of these mice were thickened presumably due to the increased pressure and there is significant myocardial hypertrophy present, which is what is found in human hypertension. Thus, the increase GRK2 in human hypertension is critically important to attack therapeutically since changing only this parameter leads to significant hypertension.

This is the first mouse model describing the vascular SMC targeting expression of a transgene that has altered the phenotype of the animal. These experiments illustrate that increasing GRK2 (βARK1) expression in VSM causes enhanced chronic in vivo resting blood pressure with a concomitant increase in arterial wall thickness and cardiac hypertrophy. The transgenic animals of the invention have attenuated βAR-mediated signalling in vascular smooth muscle cells and in vivo vasodialation. These mice represent a novel model of elevated blood pressure that permit study the role of GRK2 in the pathogenesis of hypertension and vascular hypertrophy. Moreover, they provide insight into the fact that targeting βARK1 or GRK2 activity is a novel therapeutic target and strategy for reducing systemic high blood pressure and treating hypertension.

It will be appreciated from the foregoing that the animals described herein, and cells derived therefrom, can be used in a variety of screening protocols designed to identify therapeutically effective agents. More specifically, these animals, and cells derived therefrom (e.g., vascular smooth muscle cells), can be used to assess the ability of test compounds to reduce expression and activity of GRK2. Compounds identified (or identifiable) using such screens can be used in therapeutic strategies designed to treat hypertension. The compounds can be formulated as pharmaceutical compositions that include a pharmaceutically acceptable diluent or carrier. Such compositions can be sterile, in dosage unit form and/or suitable for administration by injection, or otherwise. Optimum dosing regimens can be readily established by one skilled in the art.

Certain aspects of the invention can be described in greater detail in the non-limiting Examples that follows.

### EXAMPLE 1

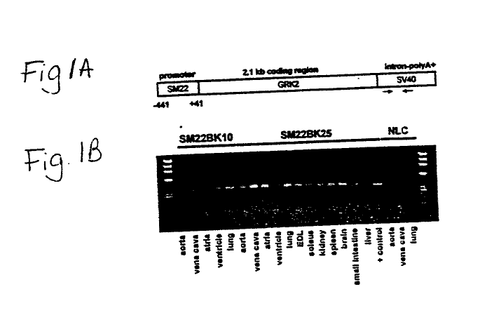

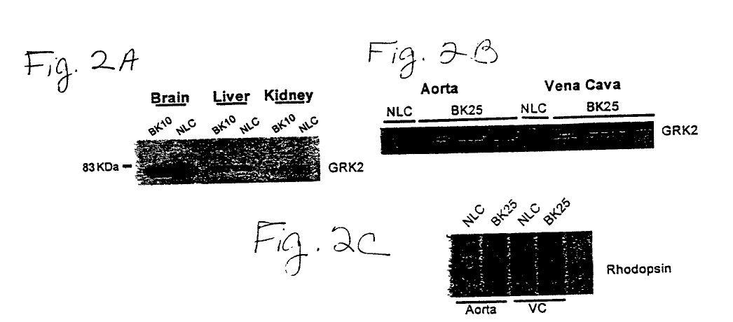

In order to understand the mechanism of GPCR signaling and desensitization by GRK2, transgenic mice overexpressing this GRK were generated by using the SM22α promoter. To generate mice with targeted VSM expression, amplification, using standard PCR techniques, of a 481 bp portion of the SM22α promoter from mouse genomic DNA that spans from −441 to +41 (relative to transcription start) was effected. This portion of the promoter is necessary and sufficient to direct robust transcription in VSM but not in non-vascular tissues both in vivo and in vitro (Kim et al, Mol. Cell Biol. 17:2266-2278 (1997); Solway et al, J. Biol. Chem. 270:13460-13469 (1995)). The transgene used was the full coding region of bovine GRK2 (βARK1). This SM22α-GRK2 construct also contains a poly-adenylation signal for SV40 to provide mRNA stability and aid in detection of transgene incorporation for screening purposes by slot blot analysis of genomic DNA isolated from tail-cuts (see FIG. 1A). This transgenic construct was linearized and given to the Duke University Transgenic Mouse Facility for microinjection and generation of founder lines of transgenic mice. Three founder lines have been generated for the GRK2 overexpressing mice and preliminary studies have been completed in two independent lines of the SM22α-GRK2 overexpressing mice, BK10 and BK25. FIG. 1B shows the mRNA expression of SV40, only present in the two independent positive transgenic mouse lines, BK10 and BK25 as assessed by RT-PCR but not in the non-transgenic littermate control (NLC) mice. Because the transgene expression was present in all of the different tissue types examined (vascular tissues aorta and vena cava but also other tissues including kidney, liver and lung), GRK2 protein expression was examined in kidney, liver and lung (FIG. 2A). No overexpression of GRK2 was detected in these cells of these tissues. This indicates that the mRNA expression seen by RT-PCR was likely due to overexpression of GRK2 in the blood vessels within these tissues.

Vascular tissue is limited in quantity in mice and therefore for initial protein expression characterization, primary smooth muscle cell (SMC) cultures were generated from the aorta and vena cava of 6 BK25 mice. There was a 3.8-fold overexpression of GRK2 in aorta and 4.3-fold in vena cava SMC as assessed by GRK2 expression and activity in BK25 versus NLC mice (FIGS. 2B and 2C).

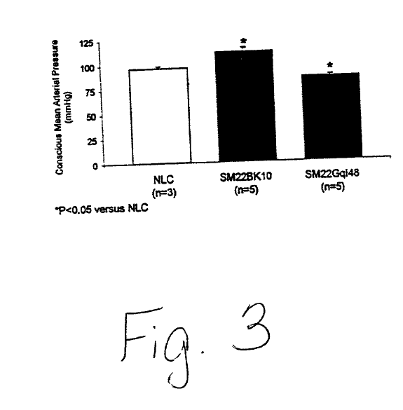

After establishing that SM22α could direct specific, robust, active VSM overexpression of GRK2 (importantly at the levels found in human hypertensive patients (Gros et al, J. Clin. Invest. 99:2087-2093 (1997)), the blood pressure of conscious mice was examined using an indwelling carotid catheter. Conscious mean arterial pressure (MAP) was increased in BK10 mice (NLC: 96±2 mmHg, n=3 versus BK10: 111±4 mmHg, n=5, p=0.04) (FIG. 3) as was diastolic pressure (NLC: 84±3 mmHg, n=3 versus BK10: 99±4 mmHg, n=5, p=0.02), whereas heart rate was unchanged. These results were also found in BK25 mice as well. Thus, in both lines of mice overexpressing GRK2 in VSM, there is conscious hypertension present.

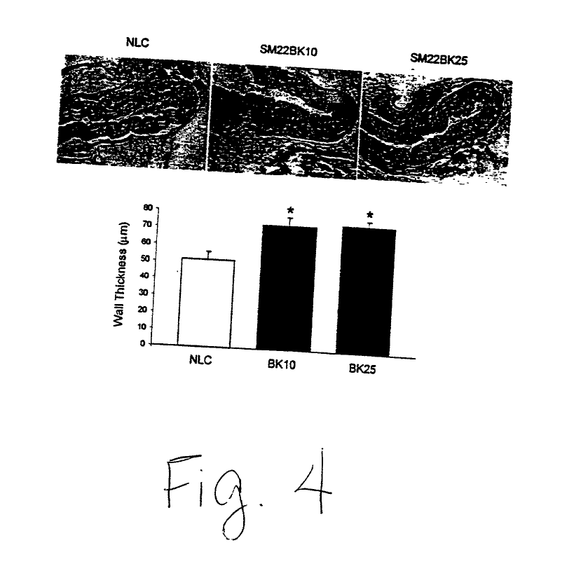

Interestingly, the chronic resting hypertension in BK10 mice led to a significantly enhanced heart weight to body weight ratio (NLC: 4.7±0.1 mg/g, n=4 versus BK10: 5.2+0.1 mg/g, n=3, p<0.01) demonstrating that these mice had cardiac hypertrophy, which can be found in human hypertension. This was also the case for the BK25 line of mice. Furthermore, aorta wall thickness was found to be increased in transgenic mice with VSM overexpression of GRK2 (NLC: 51±5 μm versus 73+5 μm in BK10 and 75+3 μm in BK25, p<0.01) (FIG. 4). These data demonstrate that GRK activity and receptor desensitization play important roles in the establishment of normal resting vascular tone and hypertrophy.

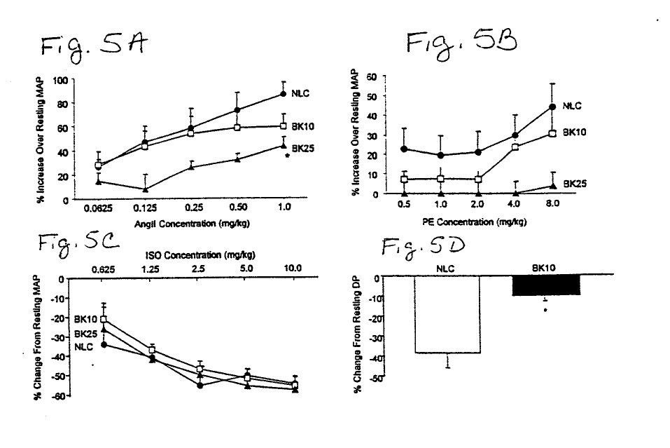

In order to begin to understand the mechanisms involved in the GRK2 overexpression-induced hypertension and the receptors that are desensitized, blood pressure responses were measured in anesthetized mice following angiotensin II (FIG. 5A), phenylephrine (FIG. 5B) or isoproterenol (FIG. 5C) infusion. The response to 1 μg/kg angiotensin II was attenuated (NLC 91+12%, n=9 versus BK25 43+8% increase over resting MAP, n=4, p=0.02), as were responses to 8 μg/kg phenylephrine (NLC: 44±11% versus 4±5%, n=4, p=0.04). MAP responses to isoproterenol were not different in GRK2 overexpressing mice as compared with NLC. However, isoproterenol has a significant effect on heart rate that could affect MAP. Therefore, diastolic pressure was examined as a rough estimate of peripheral resistance. It was found that at the lowest dose of isoproterenol tested (0.625 μg/kg), diastolic pressure was attenuated in GRK2 overexpressing mice as compared to NLC (NLC −39±8%, n=9 versus BK25: −11±3%, n=4, p=0.03) (FIG. 5D) suggesting that βAR signaling is attenuated.

### EXAMPLE 2

Transgenic Mice with Vascular Smooth Muscle Targeted Overexpression of the βAdrenergic Receptor Kinase (βARK1) are a Novel Model of Hypertension

Hypertension can reflect an imbalance between G protein-coupled receptor (GPRC) mediated vasoconstriction and vasodilation. Impairment in βAR-mediated vasodilation due to an alteration in receptor/G-protein coupling increases systemic vascular resistance and occurs in both human and animal models of hypertension. Since βARK1 expression and activity is selectively increased in hypertensive patients, it was examined whether βARK1-mediated GPCR desensitization is sufficient to increase blood pressure. A 441 bp portion of the SM22α promoter was used to target vascular smooth muscle cell (SMC) specific expression of βARK1 in transgenic mice. Parameters from two lines, BK10 and BK25 were examined. There was a 3.8-fold overexpression of βARK1 in aorta and 4.3-fold in vena cava SMC as assessed by protein expression and activity in BK25 versus wildtype (WT) mice. Conscious mean arterial pressure (MAP) was increased in BK10 mice (WT 96±2, n=3 vs BK10 111±4 mmHg, n=5, p=0.04) as was diastolic pressure (WT 84±3, vs BK1099±4 mmHg, n=5, p=0.02) whereas heart rate was unchanged. This chronic resting hypertension in BK10 mice led to a significantly enhanced heart weight to body weight ratio (WT 4.7±0.1, n=4 vs BK10 5.2±0.1 mg/g, n=3, p<0.01). Furthermore, aorta wall thickness was found to be increased in transgenic mice with SMC overexpression of βARK1 (WT: 51±5 μm vs 73±5 μm in BK10 and 75±3 μm in BK25, p<0.01). Anesthetized MAP responses to 1 μg/kg angiotensin II were attenuated (WT 91±12%, n=9 vs BK25 43±8% increase over resting MAP, n=4, p=0.02), as were responses to 8 μg/kg phenylephrine (WT 44±11% vs BK25 4±5%, n=4, p=0.04). Diastolic pressure responses to 10 μg/kg isoproterenol were also attenuated (WT −39±8%, n=9 vs BK25 −11±3%, n=4, p=0.03). Thus, signaling through several SMC GPCRs appears desensitized. In summary, increasing βAKR1 expression in vascular SMC causes enhanced chronic in vivo resting blood pressure with a concomitant increase in arterial wall thickness and cardiac hypertrophy. The mice represent a novel model of hypertension and provide insight into the role of βARK1 in the pathogenesis of this disease process.

### EXAMPLE 3

Experimental Details

Transgene construction and development of transgenic mice: A 481 bp portion of the SM22α promoter (−441 to +41 relative to transcription start) was amplified using PCR. This portion of the promoter was ligated into a previously described plasmid containing the SV40 intron poly(A+) signal (Koch et al, Science 268:1350-1353 (1995)) along with a 2070 bp fragment containing the coding sequence for bovine GRK2. The SM22α-GRK2 transgene underwent pronuclear injection. Offspring are screened by slot blot analysis of genomic DNA using a probe to the SV40 sequence. Second generation adult animals (2-12 months of age) were used for all studies.

Transgene Expression: Total RNA was extracted with RNAzol (Biotecx Laboratories, Houston, Tex.). Reverse transcription was performed using ProSTAR First Strand RT-PCR Kit (Stratagene, La Jolla, Calif.). The SV40 portion of the transgene was amplified using primers 5′-TGAATGGGAGCAGTGGT-3′ and 5′-TATGCCTGTGTGGAGTAAGAA-3′ at a concentration of 300 nM, 1.5 mM MgCl₂, 200 μM dNTPs and 1 Unit Tfl polymerase (Promega, Madison, Wis.). Reaction conditions were: 94° C. 5 min, 94° C. 30s, 65° C. 30s, 72° C. 45s, 72° C. 5 min. PCR products were run on a 1.2% agarose-TAE gel and visualized with ethidium bromide staining. Protein expression of transgene was determined using protein immunoblotting as described, from cell extracts using polyclonal GRK2 antibodies (Koch et al, Science 268:1350-1353 (1995)).

Determination of Protein Expression: 10 aorta were pooled and the VSM layer of the aorta was enzymatically digested as described below. Frozen samples were pulverized using a tissue smasher and homogenized as previously described (Koch et al, Science 268:1350-1353 (1995)). To determine GRK2 protein expression, 40 μg of protein was resolved on a 12% SDS-PAGE gel and transferred to nitrocellulose. The membrane was immunoblotted for GRK2 using the appropriate primary (Santa Cruz, Calif.) and secondary antibodies and standard chemiluminescent detection (ECL, Amersham).

Cell Culture: Primary cultures of VSM from thoracic aortas and vena cava were obtained and cultured as described (Chen et al, J. Biol. Chem. 270:30980-30988 (1995)). To determine adenylyl cyclase activity, cultured cells were grown to four days post-confluence. Cells were then labeled overnight in 3.0 μCi/mL [³H]adenine (DuPont/NEN) in medium 199 and then preincubated in medium 199 with 10 mM Hepes and 1 mM 3-isobutyl-1-methylxanthine (IBMX) for 30 min. Cells were then stimulated with the appropriate concentration of isoproterenol or 10 mM forskolin for 15 minutes. Following incubation, cAMP was determined by anion exchange chromatography and a percent incorporation of the total [³H] uptake was calculated.

MAPK Activity: To study MAPK activity, cells were stimulated with isoproterenol at the described concentration for 5 minutes. Cells were then harvested and homogenized in ice-cold RIPA buffer with 1 mM sodium orthovanadate (Iaccarino et al, Proc. Natl. Acad. Sci. USA 96:3945-3950 (1999b)). MAPK activity as assessed by phosphorylated protein levels was performed by activity assays as previously described (Iaccarino et al, Proc. Natl. Acad. Sci. USA 96:3945-3950 (1999b)). Protein immunoblotting for the activated phosphorylated forms of ERK1/2 and JNK1/3 (New England Biolabs) was normalized to total MAPK levels using antibodies specific to total ERK1/2 and JNK1 (Santa Cruz Biotechnologies, California).

In Vitro Physiology: Aorta were dissected and cut into 2.5 mm rings. Rings were then placed in a 37° C. chamber (Kent Scientific) containing Krebs-Hensileit buffer and bubbled with 95% O₂/5% CO₂. Two stainless steel wires were placed through each ring and one wire is attached to a fixed end. The other was attached to a force transducer and contraction is measured by force displacement (PowerLab, ADInstruments, Mountainview, Calif.). Responses to various agonists were tested in the presence and absence (mechanically scraped using a thin wire or chemical inhibition of nitric oxide synthase using 100 μM L-NAME) of endothelial cells. For isoproterenol responses, pretension was established at 60% of the maximum phenylephrine response (3×10⁻⁷M PE).

In vivo Physiology: Mice were anesthetized with ketamine (100 mg/kg body weight) and xylazine (5 mg/kg body weight). Subsequently, a flexible plastic catheter (flame-stretched PE50 tubing) was placed in the left carotid artery to monitor arterial pressure and tunneled subcutaneously to exit at the nape of the neck. The catheter was then flushed with 100 μL of heparinized phosphate-buffered saline (PBS) (30 U/mL), sealed and attached to the skin between the scapulae. Twenty-four hours later, blood pressure measurements are recorded from awake, unrestrained mice. Intra-arterial blood pressure is recorded continuously through the carotid catheter using PowerLab (ADInstruments, Mountainview, Calif.) data acquisition and software. In both anesthetized and awake animals, systolic (SBP) and diastolic blood pressure (DBP), and heart rate were measured. Mean arterial pressure (MAP) was calculated as DBP+⅓(SBP- DBP). The recordings from animals in each experimental group were then integrated and averaged. To analyze acute blood pressure responses, a second catheter was placed in the jugular vein to infuse agonists. Immediately following an equilibration period, mice received a bolus injection at 2-5 min intervals.

Histology: First, mice were sacrificed and aorta wall thickness was determined by perfusing for 10 minutes at 100 mmHg with PBS. Subsequently, mice were whole-body perfusion fixed at 100 mmHg using 10% neutral-buffered formalin. The aorta were then removed and fixed for another 4 h in 10% neutral-buffered formalin at 4° C., rinsed and stored in 70% ethanol. Aorta were paraffin embedded and sectioned on a cryostat. The resulting sections were stained with a modified Verhoeff VanGieson/Masson's trichrome stain.

Results

In Vivo VSM-Specific GRK2 Targeting: To generate mice with targeted VSM expression of the GRK2 transgene, we amplified a 481 base pair portion of the SM22α promoter from mouse genomic DNA that spans from −441 to +41 relative to the transcription start site. This portion of the promoter is necessary and sufficient to direct robust transcription in VSM but not in non-vascular tissues as shown in both in vitro and in vivo marker gene studies (Solway et al, J. Biol. Chem. 270:13460-13469 (1995), Kim et al, Mol. Cell Biol. 117:2266-2278 (1997)). The complete reading frame of bovine GRK2 was ligated to this SM22α promoter and transgenic mice were generated as described (Koch et al, Science 268:1350-1353 (1995)). Two independent lines of SM22α-GRK2 transgenic mice were established, SM22α-GRK2-10 and SM22α-GRK2-25. No gross phenotypic changes or unusual neonatal mortality were observed in these transgenic mice as compared to non-transgenic littermate control (NLC) mice. Second generation adult animals 2-4 months of age were used for most studies.

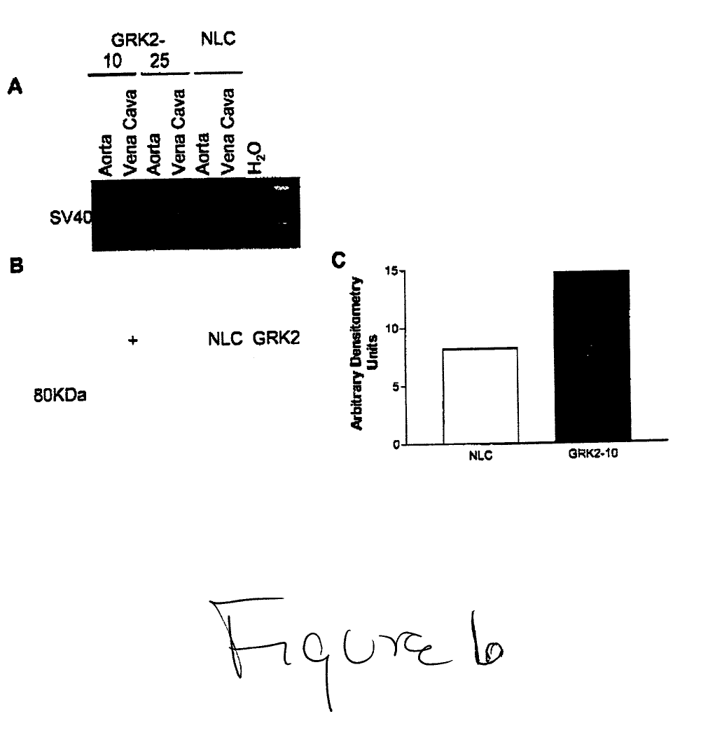

To assess mPNA transgene expression in the two lines of SM22α-GRK2 mice, RT-PCR was utilized. Positive expression was seen in only the transgenic GRK2 lines in both aorta and vena cava but not in samples from NLC mice (FIG. 6A). To assess GRK2 protein expression, we harvested aorta from 10 mice each for NLC and GRK2-10 mice. These aorta were pooled and enzymatically digested to isolate the smooth muscle layer. Protein was then extracted and 40 μg of protein was resolved on a 12% SDS-PAGE gel and transferred to nitrocellulose and immunoblotted for GRK2. As shown in the representative autoradiograph (FIG. 6B) and histogram (FIG. 6C), there was an approximately 80% increase in GRK2 expression in GRK2-10 mice as compared to NLC mice. The level of GRK2 protein expression was also examined in heart, liver, kidney and brain and no differences in the level of GRK2 expression were detected between NLC mice and the vascular transgenic mice (data not shown). Therefore, these two lines of transgenic mice appear to have vascular-specific GRK2 overexpression of ˜2-fold.

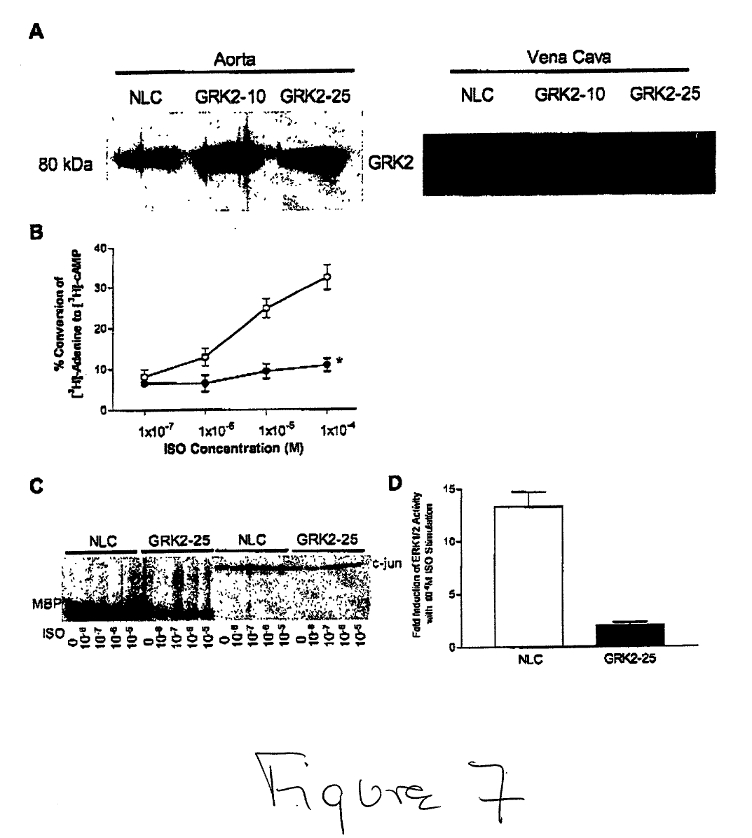

β-AR uncoupling in VSM: To study the signaling implications of vascular GRK2 overexpression primary smooth muscle cells cultured from aorta and vena cava of SM22α-GRK2 mice was generated. Aorta VSM cells, as well as venous VSM cells from SM22α-GRK2 mice had an approximate 3-fold overexpression of GRK2 (FIG. 7A). These levels of overexpression seen in culture are consistent with in vivo expression results described above and perhaps represent a more accurate measure of GRK2 levels since cultures of aorta and vena cava VSM cells are pure. Cell extracts from both lines of SM22α-GRK2 transgenic mice (GRK2-10 and GRK2-25) had almost identical transgene expression as determined by Western blots (FIG. 7A) that also resulted in a similar fold increase in VSM GRK activity as demonstrated by the ability of extracts from these cultured cells to phosphorylate the in vitro substrate rhodopsin.

To determine if enhanced expression and activity of GRK2 seen in cultured VSM cells from SM22α-GRK2 mice could exert an effect on signaling, intracellular cAMP accumulation in cultured aortic VSM cells in response to the βAR agonist isoproterenol was examined. Intracellular cAMP accumulation in cells from SM22α-GRK2-25 mice showed significant desensitization as indicated by the minimal increase in cAMP generation in response to isoproterenol as compared to cAMP generation induced by isoproterenol in VSM cells cultured from NLC mice (FIG. 7B). Similar results were observed in cells from SM22α-GRK2-10 mice. Thus, it appears that increased GRK2 overexpression in the VSM of SM22α-GRK2 mice leads to enhanced desensitization and uncoupling of βARs demonstrating the potential for exerting an in vivo physiological effect on βAR as well as other GPCR mediated signaling.

As a second assay to measure βAR mediated signaling in VSM cells from GRK2 transgenic and NLC mice, we investigated MAPK activity after βAR stimulation. Two MAPKs were studied, extracellular-regulated kinase (ERK1/2 or p42/p44 MAPKs) and c-jun kinase (JNK). FIG. 7C is a representative autoradiograph of the ability of immunoprecipitated ERK1/2 or JNK1/3 to phosphorylates their in vitro substrates myelin basic protein (MBP) or GST-c-jun, respectively, after the addition of different doses of isoproterenol to aortic VSM cells from NLC and SM22α-GRK2-25 mice. As shown, there is a dose dependent increase in the phophorylation of MBP in NLC cells, whereas phosphorylation is significantly attenuated in aorta VSM cells from GRK2 transgenic mice. This was also the case for the activity of JNK1/3 and its ability to phosphorylate GST-c-jun (FIG. 7C). Studies were also performed involving immunoblotting for the active phosphorylated form of ERK1/2. This data was normalized to total ERK levels following stimulation with 10⁻⁵M isoproterenol. There was a robust increase in induction of phosphorylation status of ERK1/2 in NLC VSM cells whereas this activation was virtually abolished in SM22α-GRK2-25 VSM cells (FIG. 7D). Thus, like βAR-mediated adenylyl cyclase activation, MAPK activation is significantly attenuated in VSM cells isolated from GRK2 transgenic mice demonstrating that GRK2 overexpression does have significant effects on GPCR signaling.

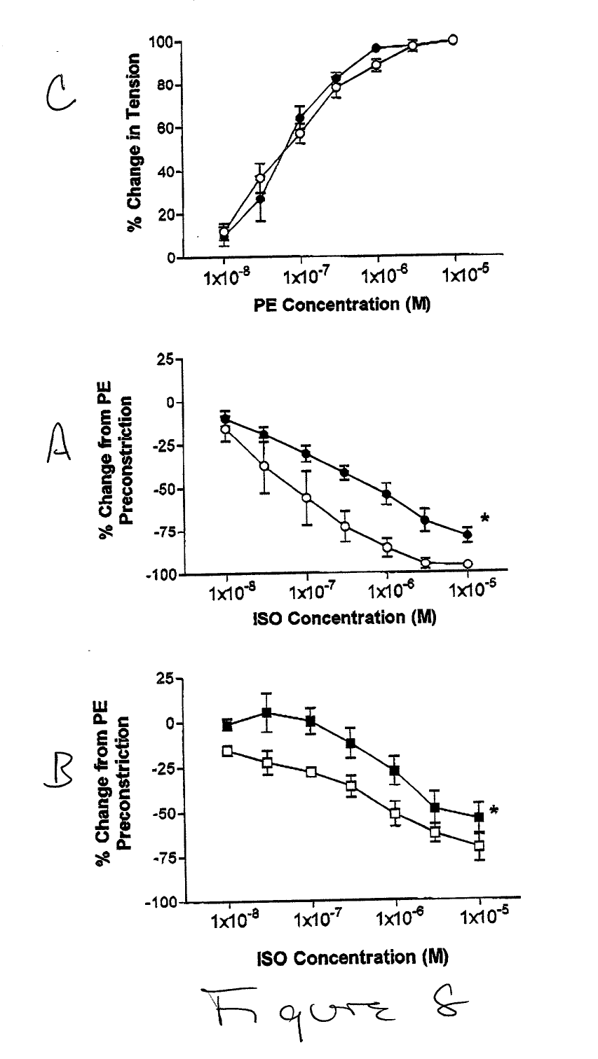

Pharmacology: To analyze the effect of VSM GRK2 overexpression independent of autonomic influences on the peripheral vasculature, responses of isolated thoracic aorta ring segments to the GPCR agonists isoproterenol (βAR) and phenylephrine (β₁AR) were examined. Endothelial cell presence was first verified by relaxation responses to 10⁻⁵M acetylcholine. Interestingly, phenylephrine-mediated constriction of aorta rings from both the NLC and SM22α-GRK2-25 mice were similar (FIG. 8A) indicating that GRK2 overexpression does not appear to alter β₁AR mediated signaling and in vivo function. Accordingly, pretension for the βAR studies was established using 3×10⁻⁷M phenylephrine, which corresponded to 60% maximum phenylephrine stimulation in both NLC and transgenic mice. VSM GRK2 overexpression was sufficient to significantly attenuate βAR-mediated vasodilation in the presence of endothelial cells (FIG. 8B). Release of nitric oxide from endothelial cells in response to isoproterenol stimulation can contribute to the βAR-mediated vasodilation therefore, aorta rings were also preincubated in 100 μM L-NAME, the nitric oxide synthase (NOS) inhibitor, for 20 minutes. Following inhibition of NOS and relative endothelial cell influence, GRK2 overexpressing mice still exhibited an attenuated response to isoproterenol as compared to NLC (FIG. 8C). Importantly, results were also similar if the endothelial cells were mechanically scraped using a thin wire as opposed to L-NAME pre-treatment. Our results indicate that approximately 40% of the NLC βAR-mediated vasodilation is due to βAR on endothelial cells and this response is preserved in GRK2 overexpressing mice (FIGS. 8B and C). Interestingly, the smooth muscle-mediated component to the vasodilation is almost completely abolished at the lower concentrations of isoproterenol and is only apparent at the higher doses (FIG. 8C).

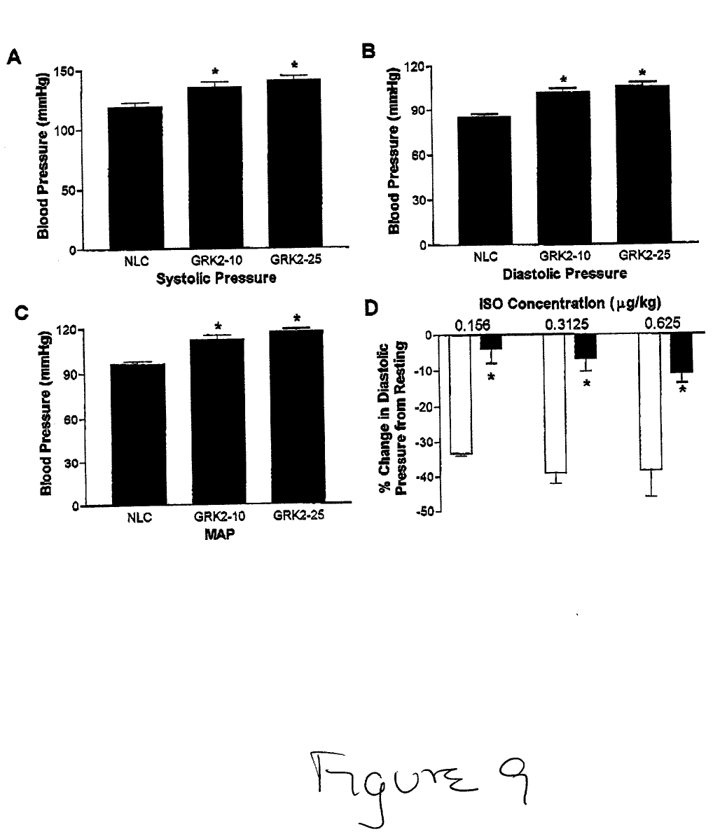

Hypertension in mice with VSM GRK2 overexpression: To investigate the effect of GRK2 on in vivo vascular function, the blood pressure of conscious mice was examined using an indwelling fluid-filled carotid artery catheter. Conscious systolic arterial pressure was increased in SM22α-GRK2-10 and GRK2-25 mice compared to NLC's (NLC, 118±4 mmHg (n=9) vs. SM22α-GRK2-10, 134±5 mmHg (n=7) and GRK2-25, 140±4 mmHg (n=5), p<0.05) (FIG. 9A) as was diastolic pressure (NLC 85±2 mmHg (n=9) vs. SM22α-GRK2-10 101±3 mmHg (n=7) versus GRK2-25 105±3 mmHg (n=5), p<0.05) (FIG. 9B). Mean arterial pressure (MAP) was also significantly increased by ˜20% in both lines of vascular GRK2 overexpressing mice as compared to NLC animals (FIG. 9C). Heart rate was unchanged between the different groups.

To understand the mechanisms involved in the GRK2-induced hypertension in these transgenic mice, in vivo blood pressure responses in anesthetized mice to isoproterenol were measured. Similar to the data obtained in conscious animals (FIGS. 9A-C), resting MAP was significantly increased in anesthetized SM22α-GRK2-10 (89.3±7.4 mmHg, n=9) compared to NLC mice (60.6±3.2 mmHg, n=10) (p<0.05, unpaired t-test). First, a significant attenuation in the βAR-mediated decrease in MAP elicited by isoproterenol was found with the overexpression of GRK2 in VSM. This depression was most significant at the lowest doses of isoproterenol (i.e. at 0.156 μg/kg isoproterenol: −30±2.8 mmHg decrease from resting MAP, n=3 versus SM22α-GRK2 −4.7±3.2 mmHg, n=3). More importantly, diastolic pressure, which is an essential component of peripheral vascular resistance, was also significantly attenuated in SM22α-GRK2-10 and GRK2-25 mice as compared to the responses in diastolic pressure in NLC mice (FIG. 9D). Thus, in SM22α-GRK2 mice, there is a significant impairment in βAR-mediated diastolic blood pressure that correlates with the attenuated vasorelaxation described above in FIG. 8, resulting in impaired MAP responses to catecholamines with the overall result hypertension.

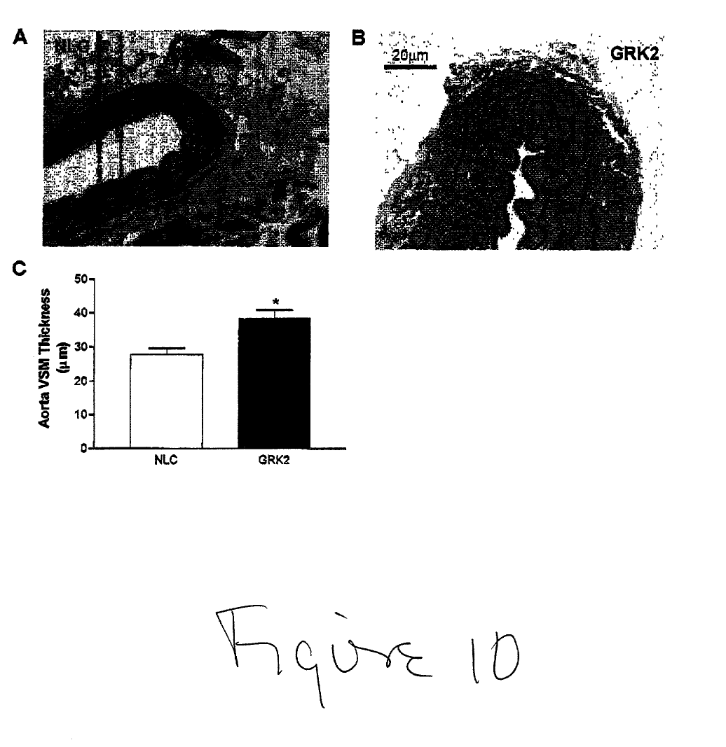

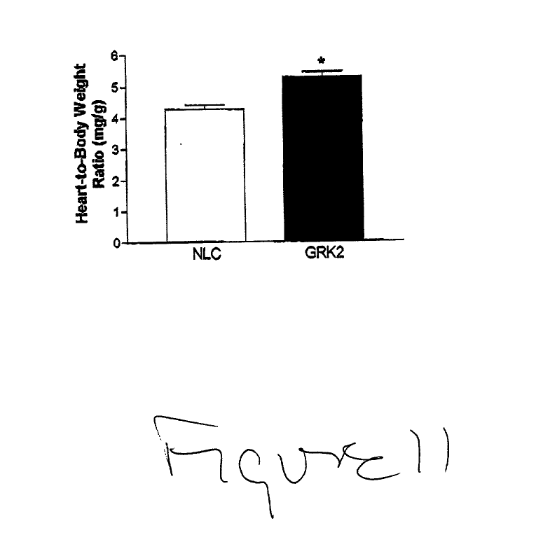

As further evidence that SM22α-GRK2 transgenic mice have a phenotype of hypertension, we found aortic vascular wall thickness to be significantly increased by ˜30% in aortas isolated from perfusion-fixed GRK2 overexpressing mice (FIG. 10). Importantly, this vascular hypertrophy is apparent in only the VSM layer and there does not appear to be any differences in collagen and elastin deposition between the NLC and SM22α-GRK2 lines (FIG. 10). Furthermore, we found that SM22α-GRK2 transgenic mice had significant myocardial hypertrophy probably as a result of the increased MAP. This was found by both an increase in heart-to-body weight ratios (FIG. 11A) as well as significant mRNA expression for brain natriuretic peptide (BNP) in the ventricles of SM22α-GRK2 mice (FIG. 11B).

All documents cited above are hereby incorporated in their entirety by reference.

1. A non-human transgenic animal that overexpresses GRK2 in vascular smooth muscle cells thereof, and cells derived from said animal.

2. The animal according to claim 1 wherein said animal is a rodent.

3. The animal according to claim 2 wherein said animal is a mouse.

4. A method of screening a test compound for antihypertensive activity comprising administering said compound to said animal according to claim 1 and assessing the effect of said compound on GRK2 activity in vascular smooth muscle cells of said animal, wherein a compound that reduces GRK2 activity is an anti-hypertensive.

5. A compound identifiable according to the method of claim 4.

6. A method of treating hypertension in a mammal comprising administering a compound identifiable by the method according to claim 4 in an amount sufficient to effect said treatment.
